(19) United States
(12) Patent Application Publication
(10) Pub. No.: US 2019/0254626 A1
(43) Pub. Date: Aug. 22, 2019

(54) GRAPHICAL USER INTERFACE FOR ULTRASOUND SYSTEM

(71) Applicant: Sonivate Medical, Inc,, Portland, OR (US)

(72) Inventors: Scott Corbett, Portland, OR (US); Austen Angell, Portland, OR (US); James T. Hatlan, Portland, OR (US)

(73) Assignee: Sonivate Medical, Inc., Portland, OR (US)

(21) Appl. No.: 16/272,181

(22) Filed: Feb. 11, 2019

Related U.S. Application Data
(60) Provisional application No. 62/634,132, filed on Feb. 22, 2018.
(60) Provisional application No. 62/634,101, filed on Feb. 22, 2018.

(51) Int. Cl. A61B 8/00

(52) U.S. Cl. CPC A61B 8/4427; A61B 8/467; A61B 8/461; A61B 8/4245

(57) ABSTRACT

Disclosed are portable ultrasound systems for use in trauma triage and assessment, such as for performing an eFAST exam. The systems include a tablet-based or mobile phone-based graphical user interface (GUI).

### CROSS REFERENCE TO RELATED APPLICATIONS

This application claims priority to U.S. Provisional Patent Applications 62/634,132 entitled Wearable Ultrasound Probe and System, and 62/634,101, entitled Graphical User Interface for Ultrasound System, and U.S. Design Application 29/660,815 Application entitled Graphical User Interface for Ultrasound System all of which are hereby incorporated by reference in their entireties for all purposes.

### STATEMENT OF GOVERNMENT SUPPORT

This invention was made with government support under contract number W81XWH-17-C-0024 awarded by The United States Army Medical Research and Material Command.

### TECHNICAL FIELD

Embodiments relate to medical imaging technologies, and more specifically, to user interfaces for portable ultrasound technologies

### BACKGROUND

Many trauma patients have injuries that are not apparent on the initial physical examination. For example, patients with penetrating cardiac trauma, blunt or penetrating abdominal trauma, or chest trauma may have sustained life threatening injuries without much external blood loss. Without rapid assessment of internal bleeding, these injuries may be overlooked in the initial assessment of a patient, and appropriate treatment may be delayed.

Ultrasound imaging can be used to identify the accumulation of intraperitoneal or pericardial free fluid and/or collapsed lung in trauma patients. Emergency physicians in the United States began using bedside or Point of Care (POC) ultrasound imaging of trauma patients in the 1980's. Ultrasound imaging has since become the initial imaging test of choice for trauma care in the United States and is part of the Advanced Trauma Life Support protocol developed by the American College of Surgeons.

POC ultrasound imaging of trauma patients consists of either the Focused Assessment using Sonography in Trauma (FAST) exam or the extended FAST exam (eFAST).

Ultrasonic eFAST examination provides a universally-accepted triage and trauma assessment tool. The eFAST exam is quicker and less expensive compared to computative topography (CT) imaging, and thus can provide vital information without the time delay caused by radiographs or CT imaging. An experienced user can conduct an eFAST examination in five minutes.

An eFAST examination involves seven to nine separate scans. Each scan requires the operator to move the probe to a different area of the body, adjust the operation of the probe, and acquire and interpret scans of the relevant physiology. Some scans should be performed with an entirely different probe. The eFAST exam typically requires two probes: one with a low frequency ultrasound array, i.e., 1-5 MHZ, for deep abdominal scans, and a probe with a high frequency ultrasound array, i.e., 5-13 MHZ, for shallow scans, such as to detect pneumothorax or collapsed lung. Low frequency phased arrays have the additional advantage of being able to minimize visual interference from ribs, and high frequency arrays provide greater image clarity for near field viewing as described further below. For many portable or cart-based ultrasound systems, an operator must disconnect one probe, connect another probe, adjust the system to accommodate the change in probe, position the probe at the relevant area of the body, and acquire and interpret the image. The majority of hand-held ultrasound systems requires two different probes to conduct an eFAST examination—one of each high and low frequency.

The number of scans and sequence in which eFAST scans are performed is subject to the personal preference of the clinician performing the scan, informed by the clinical impression of the patient. A clinician who suspects collapses lung or pneumothorax will likely begin the examination with thoracic scans, while a clinician who suspects abdominal trauma may begin the examination in the pelvic region.

Battlefield medics have an urgent need for a fast and effective way to triage individuals who have sustained traumatic injuries. The eFAST exam would provide battlefield medics with an important triage tool. However, battlefield medics are typically inexperienced or novice ultrasound operators. Conventional equipment is designed for the use of operators with extensive experience and training in the use of ultrasound. It provides little structure or guidance in order to afford the operator with the opportunity to conduct the test in accordance with his or her preferences and impressions of the patient as informed by clinical judgment. This lack of structure or guidance does not provide an inexperienced operator with necessary support. Novices users and even those who use ultrasound infrequently typically find conventional controls and/or user interfaces to be counterintuitive and unhelpful. This lack of structure or guidance is not a problem in the context of a clinic or hospital, where personnel having specialized training and experience operating ultrasound systems are readily available. But battlefield medics must triage patients with the skills they have, often under exceptionally stressful circumstances.

Bulky equipment cannot be carried into the field without compromising the mobility and safety of the operator. Switching back and forth between probes and adjusting the machine accordingly make additional demands on a medic who is fully occupied with triaging and caring for patients and responding to the demands of a battlefield environment.

Emergency responders who are not battlefield medics also must accurately and rapidly triage patients under extraordinarily demanding and difficult circumstances including but limited to mass shootings, natural disasters, etc. eFast examinations would also be of value to emergency responders, but many of the same problems with conventional systems make their use by emergency responders in the field impractical.

### BRIEF DESCRIPTION OF THE DRAWINGS

FIG. 1 is a lateral sectional view of a wearable, finger-mounted ultrasound probe, illustrating the angle between the longitudinal axis and the first array, in accordance with various embodiments;

FIG. 2 is a lateral sectional view of the finger-mounted ultrasound probe of FIG. 1, illustrating the angle b′ between the longitudinal axis and the axis b of the second array, in accordance with various embodiments;

FIG. 3 is a lateral sectional view of the finger-mounted ultrasound probe of FIG. 1, illustrating the angle c′ between the axis a of the first transducer and the axis b of the second transducer, in accordance with various embodiments;

FIG. 4 illustrates the angle of the finger-mounted ultrasound probe of FIG. 1 when the second transducer is being used, in accordance with various embodiments;

FIG. 5 includes several views of another example of a wearable, finger-mounted ultrasound probe, in accordance with various embodiments;

FIG. 6 is a perspective view of an ultrasound probe in accordance with various embodiments;

FIG. 6A is a perspective view of an ultrasound probe in accordance with various embodiments;

FIG. 7 is a rear perspective view of a wearable, finger-mounted ultrasound probe in accordance with various embodiments;

FIG. 7A is a rear perspective view of a wearable, finger-mounted ultrasound probe in accordance with various embodiments;

FIGS. 8-9 are images of screens that displaying a graphical user interface (GUI) for performing an eFAST examination in accordance with various embodiments; and

FIG. 10 illustrates an ultrasound system in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide graphical user interfaces (GUIs) which in certain embodiments may be used with a dual array probe and small, portable ultrasound systems by field medics to use the eFAST examination to diagnose trauma in a battlefield environment.

Conventional ultrasound systems include at least two components: a probe and a workstation. The probe contains the array or arrays of ultrasound transducer elements that convert electrical impulses to ultrasonic energy and vice-versa. Either the probe or the workstation includes front end functions, such as beam forming or creation of electrical impulses which are converted to and from ultrasonic energy by the array(s). The workstation contains a computational back-end, which processes the image data generated by the front end, a display, and a user interface including a keyboard or other means of input of user control.

These components are typically fairly large. The inclusion of beam forming technology in the probe increases the size of the probe. Even portable systems include workstations that are typically are no less than laptop computer-sized.

Additionally, in a clinical setting, multiple personnel typically are available to perform different roles, including caring for, stabilizing, and treating the patient and performing diagnostic activities including operating an ultrasound system, obtaining images, and interpreting those images. In such a setting, personnel are generally available who have received extensive training and/or have extensive experience with ultrasound, and ultrasound examinations are generally conducted by such individuals. And because other personnel are available to perform other roles, ultrasound operators are able to focus on performing ultrasound examinations.

In contrast, a first responder or military field medic must examine, support, triage, and stabilize patients in a potentially stressful environment. They do not generally have the opportunity to develop extensive expertise, training, and experience in operating an ultrasound system, obtaining images, or understanding and interpreting ultrasound images. Nor do they generally have the ability to focus exclusively on the conduct of an ultrasound examination, as other tasks as well as the challenges of their settings compete for their attention.

Additionally, conventional equipment is generally too large to be used in the field. Even a lap top-sized ultrasound system is prohibitively large for battlefield environments, where minimizing the gear a medic must carry is critical in order to protect the medic's mobility and even safety.

In order to be useable in these circumstances, a system must be compact enough to minimize its impact on a user's mobility, and it must be as simple and intuitive to use as possible in order to minimize demands on the user's attention and cognitive capacity, yet it must provide sufficient support to enable a user with a relative lack of specialized skills to effectively and efficiently conduct the examination.

To address these issues, the disclosed systems include a graphical user interface (GUI) that resides in a mobile computing device such as a phone or tablet and is specifically adapted to enable a relatively inexperienced user to perform an effective eFAST examination, even under challenging circumstances such as a battlefield environment, and even under circumstances where he or she must attend to other tasks as well. The disclosed systems may additionally include a finger-mounted, wearable probe that is specially designed to support the performance of an eFAST examination by a relatively inexperienced user, and a component that provides power and ultrasound beamforming functionality.

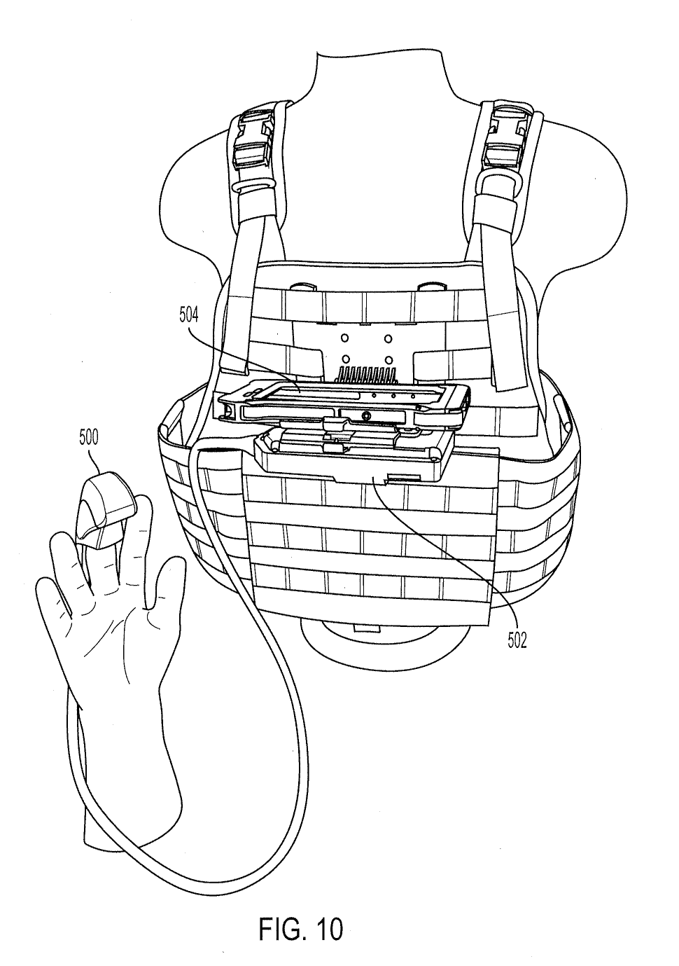

Systems disclosed herein may include a finger-mounted, wearable ultrasound probe that emits and receives ultrasonic energy, and a component that houses beaming forming technology that is electrically connected to the ultrasound probe. This component may also be worn by a user. For example, as shown in FIG. 10, this component may be worn on a user's chest, for example, by being affixed to body armor. The component is connected to transmit and receive data to and from a tablet, mobile phone, or other small computing device having a display by USB cable or other means. In various embodiments, the disclosed systems also include at least one user interface, such as a GUI, that may be displayed on a specialized ultrasound unit or on a tablet, mobile phone, or other small wireless computing device having a display, and the systems disclosed herein include at least one set of instructions stored on and executable by the ultrasound unit, tablet, phone, or other computing device. In use, execution of the instructions may cause the wearable ultrasound probe to emit and receive ultrasonic energy in accordance with one or more sets of preset parameters. In various embodiments, some processing of the data transmitted from the wearable component may be performed by an additional computing devices.

In various embodiments, the probe may be wearable. It may, for example, be adapted to be worn on a user's finger. It may include a first, low frequency phased array, and/or a second high frequency linear array.

In various embodiments, the first and second arrays may be positioned on the probe in such a way as to employ ergonomics and a user's kinesthetic sense to enhance a novel, infrequent or inexperienced user's awareness of the which array is being used. The arrays may be positioned in a manner that minimizes the change in hand position required to switch between arrays, while also providing sufficient separation between the first and second arrays to make it easy and/or intuitive for an inexperienced user to understand which array is being used (and consequently, to be able to easily interpret the resulting ultrasound images).

The two arrays may be oriented so that they have the same scan plane, which is preferably transverse to the user's finger. Having both arrays oriented in the same scan plane means that changing the array does not change the scan plane, which makes switching between arrays more intuitive for novice or inexperienced users. If the user desires to a scan plane that is transverse to the user's finger, he or she can use the array located at the tip of the finger, and can rotate his or her finger to rotate the array, a movement which is intuitive. Alternatively, he or she can hold the probe in his or her hand and rotate it.

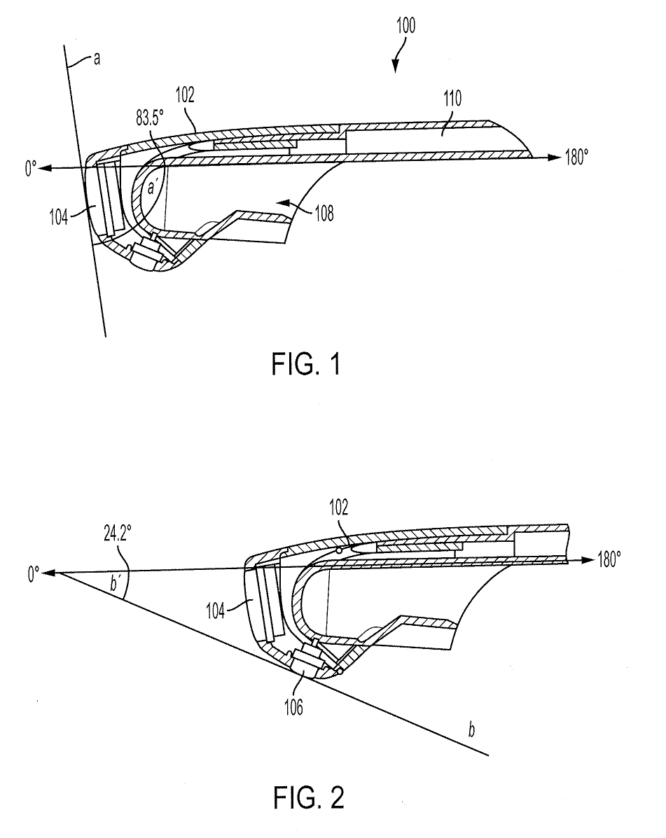

FIG. 1 is a lateral sectional view of a wearable, finger-mounted ultrasound probe, illustrating the angle between the longitudinal axis and the first array, in accordance with various embodiments. As illustrated, the wearable probe 100 may include a housing 102 having a dorsal side (top, as illustrated in FIG. 1) and a palmar side (bottom, as illustrated in FIG. 1), a proximal end (right, as illustrated in FIG. 1) and a distal end (left, as illustrated in FIG. 1), and a longitudinal axis extending therebetween and generally aligning with the longitudinal axis of the operator's finger when in use (labeled 0-180 in FIG. 1). For the purpose of this disclosure, the longitudinal axis is measured along the bottom edge of the strain relief 110, which rests on the dorsal surface of the user's finger during use.

The proximal end of the housing (closest to the operator in use) may include a finger-receiving aperture 108 so that the housing may be slid onto a user's finger. The first ultrasound array 104 may be disposed at the distal end of the housing, near the user's fingertip. The axis of the first array 104 is illustrated by line a in FIG. 1. As illustrated in FIG. 1, in various embodiments, the angle a′ between the longitudinal axis and the axis a of the first array 104 may be about 60-105 degrees, such as about 65-100 degrees, about 70-95 degrees, about 75-90 degrees, about 80-85 degrees, or about 83-84 degrees relative to the longitudinal axis.

FIG. 2 is a lateral sectional view of the finger-mounted ultrasound probe of FIG. 1, illustrating the angle b′ between the longitudinal axis and the axis b of a second array 106, in accordance with various embodiments. As illustrated in FIG. 2, the second array 106 may be disposed near the distal end of the housing 102, proximal to the first array 104. In various embodiments, the angle b′ between the longitudinal axis and the axis b of the second array 106 may be about 10-50 degrees, such as about 15-45 degrees, about 20-40 degrees, about 20-30 degrees, or about 24-25 degrees relative to the longitudinal axis.

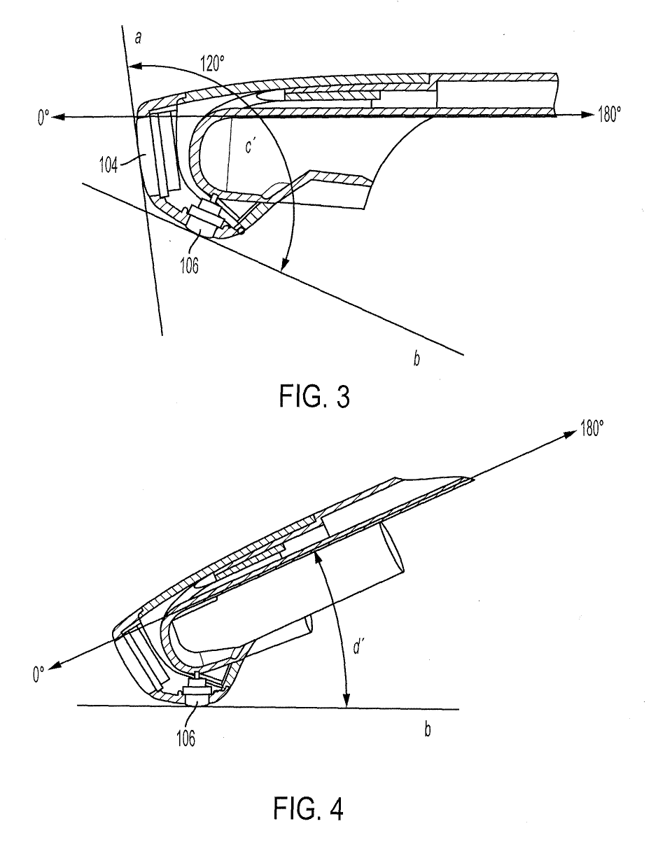

FIG. 3 is a lateral sectional view of the finger-mounted ultrasound probe of FIG. 1, illustrating the angle c′ between the axis a of the first array 104 and the axis b of the second array 106, in accordance with various embodiments. As illustrated in FIG. 3, in various embodiments, the angle c′ between the axis a of the first array 104 and the axis b of the second array 106 may be about 105-155 degrees, such as about 110-145 degrees, about 115-135 degrees, about 115-125 degrees, or about 120 degrees.

FIG. 4 illustrates the angle d′ of the finger-mounted ultrasound probe of FIG. 1 when the second array 106 is being used, in accordance with various embodiments, which is about 24 degrees. In various embodiments, the position of the finger relative to the patient during use of the probe may have a big impact on the usability of the probe, both for differentiation between the first and second arrays, and for the operator's comfort. For example, the linear array 106 needs to lie flat on the patient for the pneumothorax portion of the eFAST exam, as well as for other applications like line placements. If the angle d′ is too small, users may be unable to exert sufficient pressure on the array during imaging. Additionally, the separation between the first array 104 and the second array 106 needs to be large enough for the user to intuitively distinguish between the two array during use, but the finger angle also needs to be comfortable during use so as not to cause strain on the hand and wrist of the user. The angles and ranges defined above define a unique set of values that meet both of these conflicting needs. In various embodiments, the first and second arrays may be oriented parallel to each other. In some embodiments, both the first and second arrays may be transverse relative to the longitudinal axis.

In some embodiments, the probe may include only a single array, such as a phased array or a linear array, positioned similarly to the second array described above, albeit closer to the fingertip. In these embodiments, the angle between the longitudinal axis and the axis of the array may be about 10-50 degrees, such as about 15-40 degrees, about 20-30 degrees, or about 24 degrees relative to the longitudinal axis.

In general, probes of various shapes and architectures permit varying fields of views. For example, a curved linear array with relatively small radius of curvature permits imaging in the near field of the probe over a wide field of view. A phased array permits imaging over a wide field of view at some distance from the array, while allowing imaging through a narrow access. A linear array permits imaging over a narrower field of view, but provides good imaging of structures near the surface of the array.

One embodiment of the presently disclosed wearable probes include a phased array as the first array 104, which is positioned at the distal end of the housing, and a linear array as the second array 106, which is positioned just proximal to the first array 104. This architecture allows an operator to carry out the bulk of the eFAST exam using the first array 104, which is positioned at the tip of the finger and angled slightly toward the palmar surface (e.g., angled slightly toward the pad of the finger tip) to optimize ease of use and to afford an intuitive, ergonomic hand position during the examination. The second array 106, which is located adjacent to the first array 104, may be accessed by the operator with a slight change in hand angle for the pneumothorax-detection portions of the eFAST exam. The angle between the first and second arrays 104, 106 is optimized so that a relatively untrained operator may easily switch between arrays without confusion, while still maintaining an ergonomic hand position.

In various embodiments, the first and second arrays 104, 106 are oriented in a transverse direction, which permits a user to begin the examination with his or her hand transverse to the length of the patient's torso, which is a more natural position than parallel to the length of the patient's torso. Additionally, the combination of a straight linear array and a phased array allows the probe head profile to be minimized.

In various embodiments, the disclosed finger-mounted probes are particularly advantageous for use by users such as medics who lack specialized ultrasound expertise because the ergonomic form of the probe leverages innate hand-eye coordination and proprioception to simplify use and training by making orientation of the probe intuitive. The parallel transverse orientation of the two arrays helps prevent confusion in an inexperienced user, which is particularly important in high-stress settings, such as the battlefield. Additionally, the disclosed finger-mounted probes help keep a user's hand and arm available for other uses.

In various embodiments, the first and second arrays 104, 106 of the disclosed finger-mounted probes 100 may be electrically interconnected with a cable on a dorsal aspect of the probe 100. As illustrated in FIG. 1, a strain relief 110 may be provided to house and protect the cable. The cable may be made of flex circuit or any other electrically conductive or connective material that may be employed to electrically couple to the first and second arrays 104, 106.

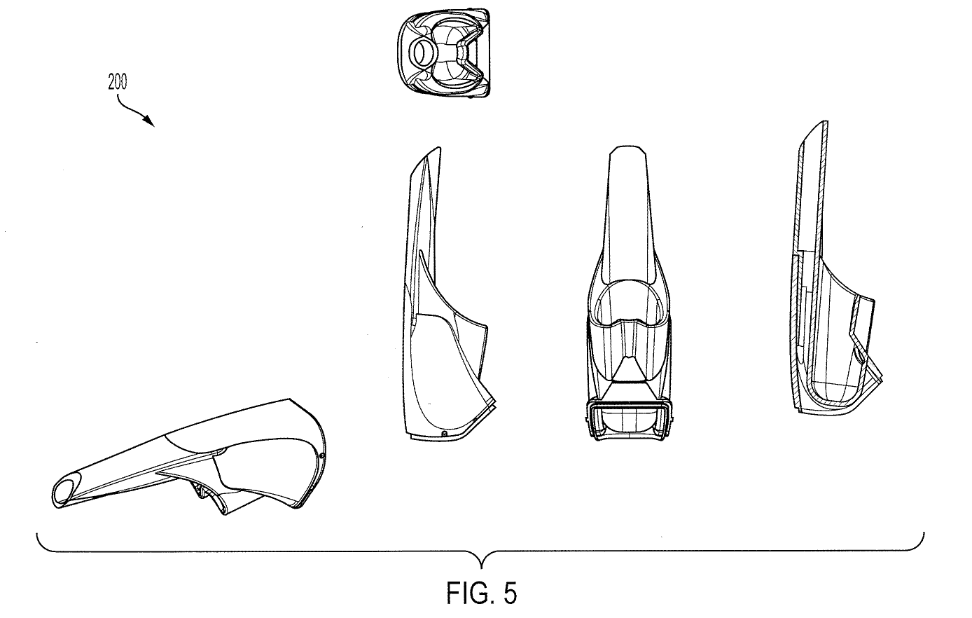

FIG. 5 is an alternate embodiment of a dual-array probe.

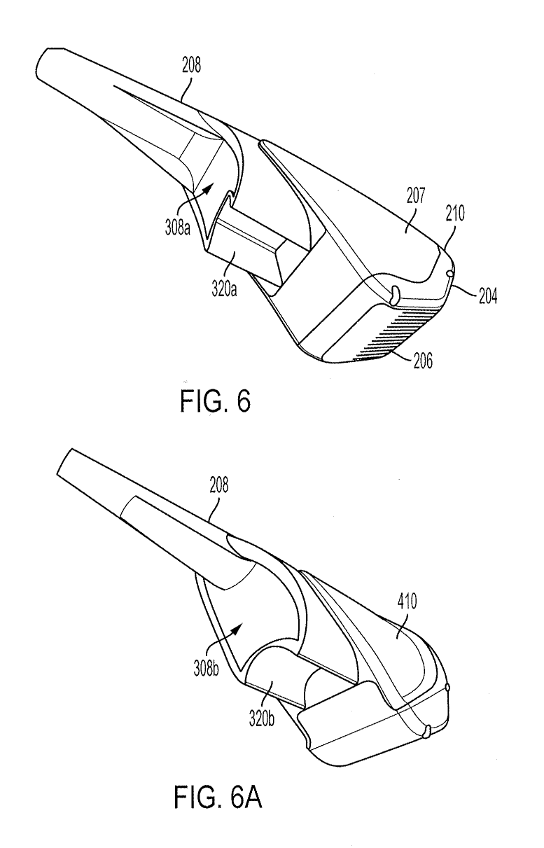

As shown in FIG. 6, probe 200 includes a first array (e.g., the phased array) 204 disposed at the distal end of a housing, and a second array (e.g., the linear array) 206 disposed adjacent the distal end, and proximal to the first array 204. The housing includes a headshell 207, strain relief 208, and nose piece 210. The housing positions the first array 204 and second array 206 in particular spatial relationships with respect to the longitudinal axis and with respect to each other, as described above. Each array includes of an array of ultrasound elements, such as piezoelectric elements or a CMUT sensor, which convert electrical impulses into ultrasonic or acoustic energy and returning ultrasonic energy into electrical impulses which can be processed into images.

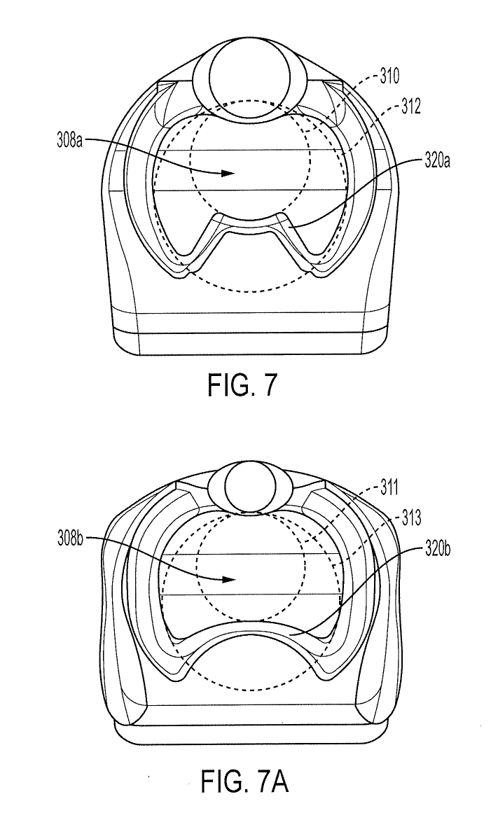

FIGS. 6 and 6A include two rear perspective views of two embodiments of a wearable, finger-mounted ultrasound probe, showing two different versions of finger-receiving apertures and finger-retaining elements, and FIGS. 7 and 7A include cross-sectional views of two finger-receiving apertures and finger-retaining elements, in accordance with various embodiments. In various embodiments, the aperture at the proximal end of the housing 308a, 308b where the operator's finger is inserted, may include a finger-retention element 320a, 320b. In some embodiments, the finger-retention element 320a, 320b may be formed from an elastomeric and/or deformable material, such that insertion of the user's finger may cause at least a portion of the finger-retention element 320a, 320b to expand or deform, thereby applying a gripping force to the finger. In various embodiments, the finger retention element may have a durometer or be made from a material having a durometer of about 30 A to 70 A, such as about 35 A to 65 A, or about 40 A to 60 A, or about 45 A to 55 A, or about 50 A. By contrast, other portions of the probe housing may be made of a harder material, such as ABS plastic, which may be about 95-115 Shore D on the hardness scale.

More specifically, in various embodiments, the finger-receiving aperture 308a, 308b may form a sleeve that includes a substantially tubular wall member formed from an elastomeric material. The sleeve may have an inner lumen sized to accommodate an average human index finger. In some embodiments, a portion of the substantially tubular wall may extend or project into the lumen to form a deformable gripping member that grips the finger. The deformable gripping member may have any of several different cross-sectional forms, such as an inward curve, arc, crease, pleat, or fold, or a more complex shape such as a combination of curves and/or folds that together form an “M” or “W” shape when viewed in cross-section. In various embodiments, insertion of a finger into the sleeve may cause the inward-facing arcuate, creased, folded, or pleated deformable gripping member to flex radially outward to accommodate the diameter of the finger. In so doing, the deformable gripping member may exert a force against the finger surface that may help retain the probe on the finger during use. As illustrated in FIG. 7, an anthropometric range of finger sizes may be accommodated by the finger-retention element 320a, 320b, from 5% (small circle 310) to 95-98% diameters (large circle 312). In various embodiments, the indented elastomeric finger-retention element 320a, 320b may distend to accommodate the large finger, yet grip the small finger. In various embodiments, the “W” shaped finger-retention element may accommodate a 95^{th} percentile finger diameter, while the “M” shaped finger-retention element may accommodate a 98^{th} percentile finger diameter.

In various embodiments, the housing may also include one or more external gripping elements, which may be a continuous softer rubber surface, or may be a discontinuous pattern of softer rubber, or may be otherwise textured, for example that may be disposed on the left and right sides of the housing, adjacent the distal end, as shown as 410 in FIG. 6A. In use, when an operator inserts an index finger into the housing, the left and right external gripping members may be positioned where the thumb and middle fingers rest, so that an operator may use the thumb and middle fingers to stabilize, rotate, and direct the probe in a desired direction/orientation to obtain a desired ultrasound image. Additionally, the external gripping elements may be used without inserting a finger into the probe, such that it may be used as a handheld probe when desired. The gripping elements enable a user to exert the necessary force and control over probe placement without the benefit of a large form factor.

As shown in FIG. 10, some embodiments of an ultrasound system in accordance with the disclosure provided herein may include three components: (1) the probe 500; (2) a wearable component 502 which can be attached to a user's body, uniform, or body armor in the region of his or her chest, and may contain a multiplexor, user interface elements, ultrasound front end processing, a beam former board, a battery, array interface board, as well as a charging board, a heat pipe, and/or a blower fan; and (3) a device for visualizing the scans and accepting user input 504 such as a tablet, mobile phone, or other wireless computing device that includes back end processing capabilities and a touchscreen display, which acts as the primary user interface. In various embodiments, the system may use a cable such as a USB Cable to connect to the tablet, phone, etc. in lieu of a wireless connection.

A beamformer emits the electrical pulses which are transformed into ultrasonic energy by the probe and used to image the patient or substrate. The beamformer originates the signal, and times it in order to focus the acoustic beam that emits from the array. The beamformer determines the amplitude and frequency of the signal. The beamformer also receives the signal and demodulates, filters, detects, and compresses the signal and converts ultrasound data into pixels, or processed image information which can then be converted to a video stream and fed to the display.

Synthetic beamforming may be used in some embodiments of the system disclosed herein. Synthetic beamforming generates ultrasound images by archiving several transmit-receive events which are then coherently summed to form a synthetic beam. The inventors of the system described herein have used synthetic beam forming to generate diagnostic quality images at up to 24 cm depth at 10 frames per second or greater with a 32 channel transmit and 16 channel receive stepped synthetic aperture. The beamformer may be located in the wearable component, rather than in the probe.

Ultrasound scanning is subject to variable parameters which are conventionally believed must be manipulated in order to enable users to optimally image structures located at various depths within a substrate such as a patient's body. Because ultrasound systems are used to visualize many different structures, ultrasound systems typically require users to adjust the system for each scan performed. Experienced ultrasound users tend to prefer systems which afford them maximum flexibility and permit them to adjust any parameter they wish.

To that end, conventional ultrasound system user interfaces typically have some or all of the following user inputs: a power switch, an ability to adjust the depth, an ability to adjust the gain, or brightness or vividness of the signal, an ability to optimize images, an ability to adjust frequency, and a zoom capability. Expert ultrasound users generally prefer the ability to adjust some or all of these parameters. Battery change indicators, screen brightness and contrast, and indications of battery power and other aspects of the system status provide information to the user. Finally, ultrasound system user interfaces typically allow users to freeze images and to save or record images or video.

The extended, Focused Assessment using Sonography in Trauma (eFAST) exam is a universally accepted triage and rapid assessment tool based on a rapid ultrasound survey of key organs, internal bleeding, and heart and lung function. The eFAST protocol involves seven to nine scans: the subxiphoid four chamber view and the parasternal long axis view of cardiac anatomy; abdominal and lower thoracic views including the upper peritoneum and Morison's pouch between the liver and right kidney and the lower peritoneum posterior to the bladder in the male and the pouch of Douglas (posterior to the uterus) in the female; right coronal and intercostal oblique views in the mid-axillary line giving coronal views of the interface between the liver and kidney; left coronal and intercostal oblique views from the posterior-axillary line producing coronal views of the spleen and diaphragm; longitudinal and transverse lower pelvic views of the bladder (male/female) and uterus (female); and anterior thoracic views of the pleural interface (to access pneumothorax) through the 3-4th intercostal space and midclavicular line.

Each scan requires a different placement of the probe on the patient's body, and conventionally, each scan is thought to require readjustment of the depth, gain, and other parameters of the ultrasound system.

The eFAST protocol does not dictate the order in which these scans are performed. These scans are typically performed in an order determined by a clinician's preference and/or determination of priority based on assessment of the patient. In other words, a clinician who suspects chest trauma may perform the subxiphoid scan first. A clinician who suspects injury to the lower abdomen may start with a pelvic view.

Each scan of an eFAST examination requires a change in the location and orientation of an ultrasound probe on a patient. This means that with each new scan, a user must remove the probe from the patient's body, recall the scans involved in an eFAST protocol, determine which scan to perform next, and place the probe in the appropriate new location on the patient's body. Additionally, two different arrays are best to perform an eFAST examination, especially for novice users such as a medic or infrequent users such as trained clinicians who use ultrasound infrequently. A phased array, emitting ultrasound waves at a lower frequency, is required to visualize and assess structures deep within the body, such as intraperitoneal or pericardial free fluid. A linear array, emitting higher frequency ultrasound waves, is required to assess structures close to the skin, such as in the detection of pneumothorax. If the next scan the user decides to perform requires a different array, the user must appreciate that fact and either adjust the system to select a different array, or, depending on the system, disconnect the previous array, connect the new array, and then adjust the system in accordance with the new array and the scan to be performed. The user must then place the probe on the patient's body and manipulate it, adjusting system parameters as needed, until a useful image is acquired. Even when the array is not changed, ultrasound system parameters, e.g., gain and depth, are often varied between scans. A clinician performing an eFAST examination must recall the appropriate depth and gain for each image and set the system accordingly. The clinician must be skilled in understanding and interpreting ultrasound images in order to determine when the correct image has been obtained and to understand the information it conveys.

Skilled practitioners in clinical settings can effortlessly remember the scans involved in an eFAST examination as well as the probes, probe placements, and system parameters necessary to perform each scan. Especially in clinical settings, such practitioners can determine which order to perform the scans, vary the orientation and placement of two different ultrasound probes and can adjust parameters as appropriate to capture adequate images with conventional equipment. However, in military contexts, first responders are military medics who must rapidly assess injured personnel without the benefits of a clinical setting. They often lack extensive training and experience with ultrasound. They are often solely responsible not just for diagnosis and triage but also for stabilizing the patient, administering first aid, and other patient care. And they must do all of this under hazardous and stressful conditions. For these reasons, eFAST examinations are typically not performed in the field by military or civilian first responders, even when fast, accurate triaging is needed to save lives.

There have been recent efforts to expand the use of ultrasound by developing systems that can be used by less experienced clinicians. Discussion of these systems is offered by way of illustration and is not meant as an admission that any of these systems are prior art. Those efforts have attempted to assist clinicians in coping with the complex task of conducting and interpreting ultrasound examinations by providing additional instruction via the user interface. For example, US Patent Publication No. 2017/0360412 teaches assistance of inexperienced ultrasound operators with the use of image recognition to analyze the images obtained and automatically generate instructions to an operator based on the images the operator obtains, such as “turn counter-clockwise.” However, the user interface is does not offer any basic guidance regarding how to perform a particular examination, and it is up to the user to use his or her expertise and experience to determine how to proceed and to sequence and perform the examination. Instructions such as those generated by such a system add complexity to an already complex task. A battlefield medic using an ultrasound system to triage is already coping with the cognitive demands of patient care and triage as well as the demands and dangers of a battlefield environment. He or she may not have the additional cognitive capacity to interpret and respond appropriately to the variable instructions generated such a system. Distraction under such circumstances could even be dangerous. Additionally, these systems are not practical in a battlefield situation because the additional processing capability they require adds weight and bulk to the equipment, slows system processing, and/or requires a robust, continuous connection to a remote system to perform the additional processing functions.

What is needed, especially by first responders including medics in battlefield settings, is a system that facilitates the use of ultrasound by less experienced operators operating under stress by simplifying the task of performing the examination rather than by further complicating it. Such a system must simplify the examination, yet still provide sufficient support to enable a distracted user with minimal training and experience to conduct the examination in a manner that yields accurate diagnostic information.

Disclosed herein is a graphical user interface (GUI) that is specific to the eFAST examination. In various embodiments of the system disclosed herein, the GUI facilitates the performance of an eFAST examination by a novice or inexperienced user by leading users through the eFAST examination in a structured manner. Such a system would be less desirable to many more experienced or expert users because it does not provide them with the flexibility necessary to freely exercise their preferences as clinicians and respond to their clinical judgment. The GUI simplifies the task of performing an eFAST examination in a manner which benefits novice and inexperienced users.

The GUI includes a plurality of active elements, referred to herein as icons and/or buttons that are responsive to an operation mode of the ultrasound system and to a record of prior user choices as set forth herein. The GUI is displayed on a screen, for example on a tablet or mobile device. An icon represents each scan performed in an eFAST examination. When that icon is touched, it automatically optimizes at least one ultrasound system parameter for that scan, for example gain, depth, or array type/frequency, in accordance with the particular requirements of that scan.

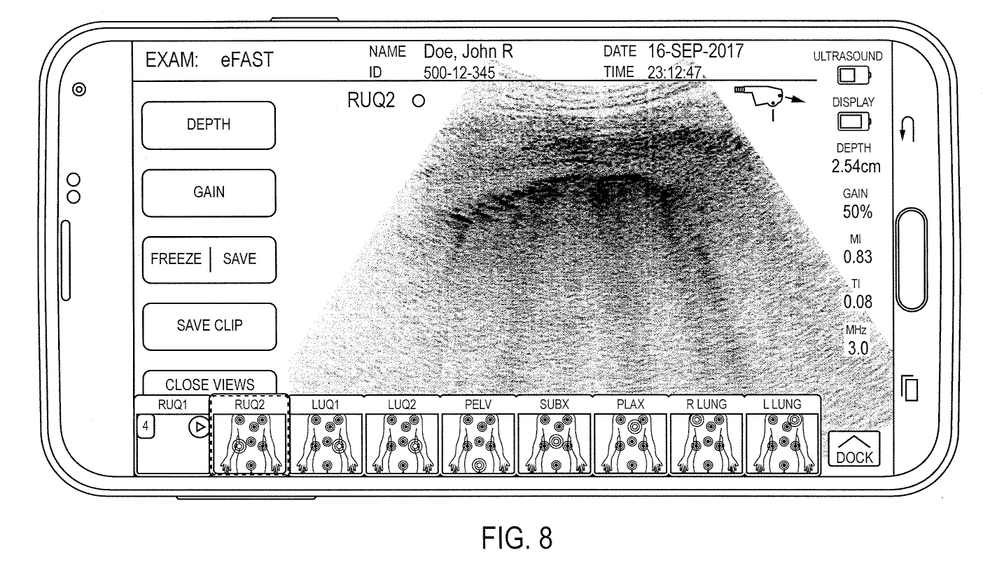

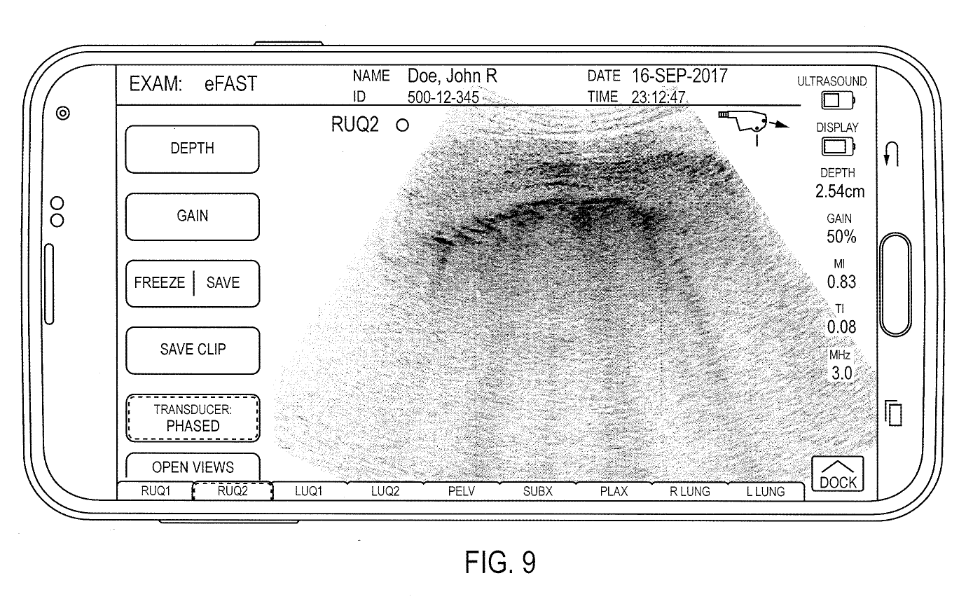

Specifically, as shown in FIGS. 8-9, the user interface presents a sequence of icons, each representing a scan to be performed in an eFAST examination, for example, as a row along the bottom edge of the screen. Each icon may be a graphical illustration of the scan. For example, as shown in FIG. 8, icons may be graphical illustrations of a human form and an indication of where on the body the probe should be placed to obtain that scan. Alternatively, as shown in FIG. 9, icons may be an abbreviation of the name of the structure scanned. The GUI presents each scan to be performed when conducting an eFAST examination serially.

Instead of leaving it up to the operator to determine how to commence and perform the examination, in certain embodiments, the GUI will direct where the examination should begin. For example, an eFAST examination performed in accordance with certain embodiments of the GUI disclosed herein will begin with the right upper quadrant view. When the examination commences, the graphical user interface presents the first scan, and each successive scan graphically in the order in which they are to be performed using icons, and in this example, indicates using an icon that the user is to proceed with this scan. A color, such as blue, indicates which scan is in progress; green for those completed and red for scans that are not yet completed. The system automatically sets the gain and depth of the probe to standardized settings that are appropriate for most patients for each scan, and the interface provides buttons that are responsive to user input and enable the user to freeze and save images and/or video for each scan. In certain embodiments, the GUI may be paired with the dual-array probe disclosed herein, and in such embodiments the system will automatically select which array is used for each scan in response to a user's selection of the scan or advancement of the system to the next scan in sequence, and will indicate which array is in use with an icon.

In accordance with certain embodiments of the system disclosed herein, a user commences with the first scan. The system automatically configures the system and probe, and in some embodiments, an icon provides the user with a reminder regarding where to position the probe. When the scan is performed, a user may cause the system to save one or more images or video clips by using one or more buttons on the GUI.

Once the user has saved a scan or clip representing that view, the system may indicate that the scan, in this example the right upper quadrant scan, has been performed, in certain embodiments by changing the color of the icon, and in other embodiments by replacing the icon with another visual, such as an image from the scan, as shown in FIG. 9. The user then advances to the next scan in the sequence or to the next desired scan. In the embodiment depicted in FIGS. 8 and 9, that scan is the next scan in the right upper quadrant view, represented as RUQ2, which in some embodiments causes the corresponding icon on the GUI to change color to indicate which scan is being performed. In some embodiments, the user chooses the next view after a clip or view from the previous scan is saved. In some embodiments, the system advances to the next scan in the sequence after the user indicates that the scan is complete. Upon advancement to the next scan, the GUI may indicate that the next scan is underway, and the system automatically sets the gain, depth, and, if a dual array probe is being used, selects the appropriate array for that scan. This sequence is repeated with each scan until the user either ends the examination or completes it.

The GUI provides images or icons representing each view, for example along the bottom edge of the screen. Each image or icon may provide visual guidance to the user regarding where on the patient's body the probe should be positioned in the next scan. When each scan is complete, the icon may change color and it may remain the new color for the duration of the examination to indicate how much of the examination has been performed. For example, when the user is performing the sixth scan in the sequence, the preceding five icons may be a color that indicates that they are complete, for example, green. The sixth icon may be a different color, for example blue, indicating that this scan is being performed, and the remaining icons will be a third color, for example red, indicating that they are still to be performed.

In accordance with some embodiments of the GUI and system disclosed herein, the scans may be performed in the following order: two right upper quadrant scans, two left upper quadrant scans, pelvic scan, subxiphoid/subcostal scan, posterior/axillary line scan, right lung, and left lung. This order may be varied without departing from the spirit and scope of the inventions disclosed herein at the discretion of the user.

The defined sequence in which the GUI leads the operator through an eFast examination enables a field or military medic to perform this examination even with limited training and experience under adverse conditions. A user interface that is specific to the eFAST exam and leads a user through the examination in a structured manner reduces the cognitive load on the operator, which is relatively unimportant in a clinical setting but critical in a battlefield setting. It reduces the need for advanced clinical judgment and does not distract the user with any extraneous information or require a user to make any extraneous decisions. A user is not required to remember unaided which scans are involved in an eFAST exam or to remember unaided where to place the probe or which array should be used.

In certain embodiments, the system automatically varies system parameters, including gain and depth, as well as automatically selecting an array, as appropriate for each successive scan. For example, for all internal bleeding scans, the phased array will be used, and it may be pre-set at about 3.0 MHZ with a footprint of about 18 mm×18 mm and a depth of about 16 cm, which have been found to be settings which will permit effective visualization of the pertinent structures on most individuals. The high frequency linear array is used for pulmonary views. It may be pre-set at about 7.5 MHZ with a footprint of about 7 mm×22 mm. The GUI may permit a user to adjust contrast or image brightness, or the GUI may set these parameters at preset values, or may vary them in accordance with information regarding ambient light obtained by sensors.

Alternatively, the GUI may permit a user to vary or adjust any or all of these pre-set parameters, or it may preclude adjustment of any or all of these parameters in order to simplify and streamline the performance of the examination.

In some embodiments, a user can vary the order of the scans by selecting the icons indicating each scan in the order in which a user prefers. The GUI will still automatically set system parameters in accordance with whichever scan is selected, and the icons may indicate through color or other means which scans have been performed.

The GUI may also include a pop up keyboard or other means that allows users to fill in patient information, comment on findings or insert associate labels or other information.

In some embodiments, the system may have a “manual” or other setting that permits a user to elect to adjust system settings in a manner more consistent with conventional ultrasound systems.

Because the GUI represents each scan as an icon, it may utilize a visual indication such as a color change to indicate which scans have been performed and which have not, an inexperienced or stressed operator can instantly orient him or herself to the task of performing the examination. The system saves the scans, and additionally in some embodiments transmits them to a field hospital, field station, base, or clinic where a physician or other clinician can assess them or help the ultrasound operator in the field to assess them. The system may automatically save the scans in a way that associates them with information regarding which view they represent, enabling the user and/or other personnel to understand and interpret them with greater speed, efficiency, and accuracy.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A graphical user interface for an ultrasound system, comprising:
a plurality of active elements responsive to an operation mode of the ultrasound system, and to a record of prior user choices;
a screen for displaying the graphical user interface; and
a user-operable element that automatically optimizes at least one setting for a first scan of an eFAST exam in response to user input, the at least one setting comprising gain, depth, or array.

2. The graphical user interface of claim 1, wherein the graphical user interface displays an image illustrating a correct probe placement for the first scan.

3. The graphical user interface of claim 1, wherein the graphical user interface displays an image illustrating which array, of a first array and a second array, is to be used for the first scan.

4. The graphical user interface of claim 1, further comprising a user-operable element that automatically optimizes at least one setting for a second scan of an eFAST exam in response to user input, the at least one setting comprising: gain, depth, or array.

5. The graphical user interface of claim 4, wherein said user-operable element visually indicates that said scan is complete in response to user input.

6. The graphical user interface of claim 4, wherein the user-operable element automatically optimizes at least one setting for a second scan when a user selects said second scan.

7. The graphical user interface of claim 1, wherein the graphical user interface comprises seven user-operable elements, each of which represents one of the scans making up an eFAST examination.

8. The graphical user interface of claim 7, wherein each said user operable elements illustrates the correct probe placement for one said scan.

9. The graphical user interface of claim 8, wherein each said user operable element changes color in response to user input indicating that the corresponding scan has been completed.

10. An ultrasound system configured for performance of an eFAST exam, comprising:
an ultrasound probe comprising two arrays, wherein said first array is a high frequency linear array and said second array is a phased array, and
a graphical user interface comprising a user-operable element that automatically activates one array in accordance with a first scan in the eFAST exam.

11. The ultrasound system of claim 10, wherein said user-operable operable element further optimizes at least one setting for a first scan of an eFAST exam, the at least one setting comprising gain or depth.

12. The ultrasound system of claim 10, wherein said ultrasound probe is a wearable probe.

13. The ultrasound system of claim 10, wherein said graphical user interface displays an image illustrating a correct probe placement for the first scan.

14. The ultrasound system of claim 10, wherein said graphical user interface displays an image illustrating which array, of a first array and a second array, is to be used for the first scan.

15. The ultrasound system of claim 10, wherein said graphical user interface further comprises a user-operable element that automatically optimizes at least one setting for a second scan of an eFAST exam, the at least one setting comprising: gain, depth, or array.

16. The ultrasound system of claim 15, wherein said user-operable element visually indicates that said scan is complete in response to user input.

17. The ultrasound system of claim 15, wherein said user-operable element automatically optimizes at least one setting for a second scan in response to user input.

18. The ultrasound system of claim 10, wherein said graphical user interface comprises seven user-operable elements, each of which represents a scans making up an eFAST examination.

19. The ultrasound system of claim 18, wherein each said user operable element illustrates a correct probe placement for a corresponding scan.

20. A method of conducting an eFAST examination, comprising:
Providing an ultrasound system comprising an ultrasound array, a computing device, and a screen, the screen displaying a graphical user interface, the graphical user interface comprising a plurality of active elements responsive to an operation mode of the ultrasound system, wherein a plurality of the active elements are graphical representations of one or more scans;
Selecting a first scan using a first said active element; and
Using the probe to obtain one or more images in accordance with the first said scan.
